(19) United States
(12) Patent Application Publication
(10) Pub. No.: US 2011/0212493 A1
(43) Pub. Date: Sep. 1, 2011

(54) PERFUSION BIOREACTORS, CELL CULTURE SYSTEMS, AND METHODS FOR PRODUCTION OF CELLS AND CELL-DERIVED PRODUCTS

(75) Inventors: Mark HIRSCHEL, Blaine, MN (US); Darrell P. PAGE, East Bethel, MN (US); Robert J. WOJCIECHOWSKI, Forest Lake, MN (US)

(73) Assignee: BIOVEST INTERNATIONAL, INC., Tampa, FL (US)

(21) Appl. No.: 13/092,836

(22) Filed: Apr. 22, 2011

Related U.S. Application Data
(63) Continuation of application No. PCT/US2009/061700, filed on Oct. 22, 2009.
(60) Provisional application No. 61/107,644, filed on Oct. 22, 2008.

(51) Int. Cl. C12N 15/64; C12M 3/00; C12N 5/071; C12P 1/00; C12N 7/00

(52) U.S. Cl. 435/91.4; 435/289.1; 435/325; 435/41; 435/235.1

(57) ABSTRACT

The present invention includes perfusion bioreactors, automated cell culture systems, and methods for production of cells and cell-derived products.

### CROSS REFERENCE TO RELATED APPLICATION

This application is a continuation of International Application No. PCT/US2009/061700, filed Oct. 22, 2009, which claims the benefit of U.S. Provisional Application Ser. No. 61/107,644, filed Oct. 22, 2008, the disclosure of each of which is hereby incorporated by reference in its entirety, including all figures, tables, and amino acid or nucleic acid sequences.

### BACKGROUND OF THE INVENTION

The anticipated growth of personalized medicine will require new paradigms for the design of therapies tailored to the needs of individual patients. The greatest challenge is expected to come in the area of cell-based therapies. Therapeutic applications of live cells hold tremendous promise and are emerging as viable treatment strategies for a wide variety of human disorders, but there remains an unmet need for safe, economical and efficient means for the ex vivo production of cells for research, clinical development, and commercialization. Current methods are expensive, labor intensive, prone to errors and contamination, suffer from the variable culture conditions, and require extensive facility infrastructure.

Autologous cell-based therapy is widely viewed as one of the most promising areas of growth in the biotechnology industry. Autologous cell therapies are accomplished by harvesting cells of one or more cell type from a patient, growing or expanding the cells in the laboratory and returning the expanded and possibly modified cells back to the patient. Applications of cell therapies include, for example, rebuilding cartilage for treatment of osteoarthritis, growing autologous skin for treatment of burns, growing fibroblasts for treatment of skin disorders, and growing muscle for treatment of cardiac disorders.

In the case of autologous cell-based therapies, each cell or cell-based product is manufactured from each patient. Manual methods for mammalian cell culture, by their nature, are prone to technician error or inconsistency leading to differences between ideally identical cultures. This becomes especially evident as more and more autologous cells are expanded for personalized therapies. Patient-specific cells, or proteins, are subject to variation, especially when scaled beyond levels that can be managed efficiently with manual methods.

In addition to being labor-intensive, the stringent requirements for segregation of each patient's materials from that of every other patient will mean that manufacturing facilities will be large and complex, containing a multitude of isolation suites each with its own equipment (incubators, tissue culture hoods, centrifuges) that can be used for only one patient at a time. Because each patient's therapy is a new and unique product, patient specific manufacturing will also be labor intensive, requiring not just direct manufacturing personnel but also disproportionately increased manpower for quality assurance and quality control functions.

Moreover, conventional approaches and tools for manufacturing cells or cell-based products typically involve numerous manual manipulations that are subject to variations even when conducted by skilled technicians. When used at the scale needed to manufacture hundreds or thousands of different cells, cell lines, and patient-specific cell-based therapies, the variability, error or contamination rate may become unacceptable for commercial processes.

Cell culturing devices or cultureware for culturing cells in vitro are known. As disclosed in U.S. Pat. No. 4,804,628, the entirety of which is hereby incorporated by reference, a hollow fiber culture device includes a plurality of hollow fiber membranes. Medium containing oxygen, nutrients, and other chemical stimuli is transported through the lumen of the hollow fiber membranes or capillaries and diffuses through the walls thereof into an extracapillary (EC) space between the membranes and the shell of the cartridge containing the hollow fibers. The cells that are to be maintained collect in the extracapillary space. Metabolic wastes are removed from the bioreactor. The cells or cell products can be harvested from the device.

The therapeutic potential of regenerative medicine has created a strong need for improved, or automated, in vitro cell culture technologies. One principle example is the creation of skin grafts to treat burn victims and chronic wound patients, such as the elderly, diabetics, or individuals who are bed-ridden or suffer from debilitating hereditary disorders.

Currently, the process for creating suitable, matched skin grafts follows a complex, labor intensive protocol executed by highly skilled technicians. Similar to biologics manufacturing in the 1970's, regenerative medicine currently suffers from a lack of automated technology to facilitate mass production. At present, keratinocytes are slowly and laboriously expanded on a culture matrix, or dish, which is coated with a suitable matrix such as collagen or chrondroitin sulfate. These cultures are then maintained by experienced technicians through use of complex growth media and cell-signaling proteins to induce cell-to-cell interaction and nurture cell growth to create an adequate graft.

A fully automated system will greatly benefit this field and significantly improve the process consistency required for therapeutic application. Similar to the device currently available for protein production, next generation bioreactors will incorporate the advantages of automated perfusion methodology to facilitate cost-effective production of therapeutic cells such as keratinocytes, for example.

Biovest International, Inc. (Tampa, Fla.) has developed and markets an automated cell culture instrument, the AutovaxID™, which utilizes hollow fiber bioreactors and advanced process control to maintain long-term, high density cultures. The instrument incorporates multiple pumps for the automated perfusion of media and growth supplements and for product harvest, feedback sensors and gas exchange capabilities for process control, and computer assisted control and record keeping designed for cGMP compliant manufacture of cells and cell-derived products. The cellulose acetate, hollow fiber bioreactors together with the AutovaxID™ system have been designed primarily for the growth of hybridoma cells and production of monoclonal antibodies. Although highly efficient for this application, the system is not optimized for growth of adherent cells or subsequent detachment and collection of viable cells as the desired product.

The use of stem cells for cell-based therapies has the potential to revolutionize the treatment of a wide range of human disorders. In recent years, enormous progress has been made in the ability to isolate and grow stem cells, and there are numerous applications currently in clinical development. Despite enormous promise, little effort has been devoted to the development of safe, economical and reproducible methods of manufacturing sufficient quantities of stem cells to meet the demands of research & development, clinical trials, or post-approval medical demands. Current methods, which rely primarily on 2-dimensional (2-D) static culture systems, are labor intensive, prone to error and/or contamination, and suffer from culture-to-culture variations, representing significant barriers to commercialization. Enabling technologies such as off-the-shelf systems for the mass production of allogeneic cells, or the personalized expansion of autologous cells, would speed the translation of cell-based treatment regimens from the research laboratory to the clinic.

### BRIEF SUMMARY OF THE INVENTION

One aspect of the present invention is a perfusion bioreactor capable of expanding cells (e.g., stein cells, progenitor cells, differentiated cells) to large numbers. Each perfusion bioreactor comprising a housing with an inlet port and outlet port, and a cell growth matrix. In some embodiments, the cell growth matrix is planar, pleated, or spirally wound around a central core. Preferably, the housing comprises a first part with an inner surface and a second part with an inner surface, wherein the first part engages with the second part (in a fluid-tight fashion) such that the inner surfaces of the first part and second part define a space occupied by the cell growth matrix (the cell growth chamber), wherein the matrix has first side and a second side, wherein the first matrix side and the inner surface of the first part define a first chamber (a first cell growth chamber or sub-chamber), and wherein the second matrix side and the inner surface of said second part define a second chamber (a second cell growth chamber or sub-chamber). Thus, the first and second chambers are separated by the cell growth matrix. Preferably, the first part of the body has an inlet port and an outlet port in fluid communication with the first chamber, and the second part of the body has an inlet port and an outlet port in fluid communication with the second chamber.

In embodiments in which the matrix is pleated, the inner surface of the first part of the body, or the inner surface of the second part of the body, or both inner surfaces, further comprise supports for supporting the pleats of the matrix. Each support can conform to the pleats on each side of the matrix. Optionally, each support has a hole that traverses the support, which permits the flow of medium through the support. Preferably, the inner surface of the first part of the body and the inner surface of the second part of the body further comprise headers which engage with each other said headers of said inner surface of said second part. Preferably, each of the headers has a hole that traverses the header, which permits the flow of medium through the header. In some embodiments, the first part and second part have two rows of headers at each end (an inner row and an outer row), wherein the inner row of headers has a hole in each header, but the headers in the outer row have a smaller number of holes to further limit flow rate.

In some embodiments, the bioreactor further comprises a central core, with the matrix wrapped around the central core.

Another aspect of the invention is an automated cell culture system for the production of cells and cell-derived products, comprising a reusable instrumentation base device incorporating hardware to support cell culture growth, and at least one disposable cell cultureware module removably attachable to the instrumentation base device, wherein the cultureware module includes a perfusion bioreactor of the invention.

Another aspect of the invention is a method for the production of cells and cell-derived products, comprising providing at least one perfusion bioreactor of the invention, introducing cells into the bioreactor, and culturing the cells within the bioreactor. Cells and/or cell-derived products may then be harvested from the bioreactor. In one embodiment, the production method involves using the perfusion bioreactor in a culture system. Thus, in this embodiment, the method comprises: providing at least one cultureware module, the module including at least one perfusion bioreactor of the invention; providing a reusable instrumentation base device incorporating hardware to support cell growth, the base device including a microprocessor control and pump for circulating cell culture medium through the bioreactor(s); removably attaching the cultureware module(s) to the instrumentation base device; introducing cells into the bioreactor(s); fluidly attaching a source of cell culture medium to the cultureware module(s); programming operating parameters into the microprocessor control; and operating the pump to circulate the cell culture medium through the bioreactor(s) to grow cells or cell products therein. Optionally, the method may further comprise harvesting the grown cells and/or cell-derived products from the bioreactor(s); and, optionally, disposing of the cultureware module(s).

One aspect of the present invention is a cell culture system for the production and expansion of cells (e.g., primary cells or cell lines) and/or cell derived products. The system includes a reusable control module housing with all of the mechanical and electronic components and disposable perfusion bioreactors that attach to the control module. This system minimizes the need for skilled technicians and more importantly, prevents the possibility of cross-contamination in a multi-use facility. As an enclosed system, the safety provided by complete segregation facilitates direct applicability to therapies or diagnoses that require autologous cell culture. This self-contained, automated cell culture device allows for simultaneously culture of numerous cell cultures within a compact facility, without the need for individual, segregated cell culture suites. The system of the present invention provides a compact sealed containment system that will enable the cost effective manufacture of cells, cell lines, patient-specific cells and cell products on an industrial scale.

The method and system of the invention can incorporate disposable cultureware, which eliminates the need for cleaning and reuse. The culture system has the stand-alone integration of a large system in a bench top device (pumps, controls, incubator, refrigerator, cultureware, etc.). The cell culture system can incorporate a barcode reader and data gathering software that, when used with an information management system (such as a manufacturing execution system or MIMS), allows for automating generation of the batch record.

The cell culture system incorporates features that greatly reduce the operator's time needed to support the operations (e.g., integrated pump cassette, pre-sterilized cultureware with pH sensors, quick-load cultureware) and designed automated procedures and apparatuses which allow the system to sequence through the operations (e.g. automated fluid clamps, control software).

The automated cell culture system creates a self-contained culture environment. The system incorporates perfusion culture with sealed, pre-sterilized disposable cultureware, programmable process control, automated fluid valving, pH feedback control, lactic acid feedback control, temperature control, nutrient delivery control, waste removal, gas exchange mechanism, reservoirs, tubing, pumps and harvest vessels. Accordingly, the cell culture system is capable of expanding cells in a highly controlled, contaminant-free manner. Cells to which this approach are applicable include transformed or non-transformed cell lines, primary cells including somatic cells such as lymphocytes or other immune cells, chondrocytes, myocytes or myoblasts, epithelial cells and patient specific cells, primary or otherwise. Included also are cells or cell lines that have been genetically modified, such as both adult and embryonic stem cells. Specifically, the automated cell culture system allows for production and harvest of cells or cell products, such as cell-secreted protein, in a manner that minimizes the need for operator intervention and minimizes the need for segregated clean rooms for the growth and manipulation of the cells. Further, the system provides a culture environment that is completely self-contained and disposable. This eliminates the need for individual clean rooms typically required in a regulated, multi-use facility. Control of fluid dynamics within the bioreactor allows for growth conditions to be adjusted, e.g., changing growth factor concentrations, to facilitate application of unique culture protocols or expansion of unique cells or cell lines. As a result, there is less variation and less labor required for consistent, reproducible production of cells for applications to expansion of autologous cells and their use in personalized medicine applications.

According to these and other aspects of the present invention, there is provided a cell culture system for the production of cells and cell-derived products including a reusable instrumentation base device incorporating hardware to support cell culture growth. A disposable cultureware module including a perfusion bioreactor is removably attachable to the instrumentation base device.

According to these and other aspects of the present invention, there is also provided a method for the production of cells and cell-derived products in a highly controlled, contaminant-free environment comprising the steps of providing a disposable cultureware module including a cell growth chamber, and a reusable instrumentation base device incorporating hardware to support cell culture growth. The base device includes microprocessor control and a pump for circulating media through the cell growth chamber. The cultureware module is removably attached to the instrumentation base device. Cells are introduced into the one or more bioreactors. A source of media is fluidly attached to the cultureware module. Operating parameters are programmed into the microprocessor control. The pump is operated to circulate the media through the cell growth chamber to grow cells or cell products therein. The grown cells and/or cell-derived products can then be harvested from the bioreactor(s). The cultureware module can then be disposed of.

These and other features, aspects, and advantages of the present invention will become more apparent from the following detailed description.

### BRIEF DESCRIPTION OF THE DRAWINGS

FIGS. 1A-1D show four views of an embodiment of the perfusion bioreactor of the invention. FIG. 1A shows a perspective view of the housing (also referred to herein as the body 6) of the bioreactor 2, which may be constructed of polycarbonate or other appropriate material. FIG. 1B is an exploded view, showing the separated parts of the body (6A, 6B), with the matrix 8 (composed of one or more materials suitable for growth of the particular cell type or types, e.g., PET, chitosan, collagen) between. FIGS. 1C and 1D are cross-sectional views of the bioreactor 2 showing how the 8 fits within the bioreactor housing 6 to create two chambers 10, 12. Each part of the body 6 has at least one inlet port and at least one outlet port. In an alternative embodiment, the parts of the body 6A and 6B cooperate to form one or more (shared) inlet port and one or more (shared) outlet ports.

FIGS. 2A-2B are cross-sectional diagrams illustrating how the bioreactor 2 shown in FIG. 1 is designed to accommodate three fluidic conditions: priming, inoculation, and recirculation (perfusion). Fluid flow is directed through the bioreactor 2 by selectively closing off inlet ports 14, 16 and outlet ports 18, 20 to accommodate these conditions. In FIG. 2A, all ports 14, 16, 18, 20 are open for system priming with media. In FIG. 2B, the tubing connecting the bottom chamber inlet 16 and the tubing connecting the chamber outlet 20 will be closed during the inoculation step. This will direct the cell suspension from the top chamber inlet 14, through the matrix 8 and out the bottom chamber outlet port 20. Upon completion of the seeding operation, the chamber outlets 18, 20 will be opened for media re-circulation, allowing for tangential flow on either side of the matrix 8.

FIG. 3 is a schematic drawing of a modified AutovaxID™ cultureware module with three of the bioreactors 2 shown in FIG. 1 arranged in parallel, and inoculation, oxygenation (recirculation), and cell recovery circuits. Thus, FIG. 3 shows a modified version of the flow path shown in FIG. 7B, which is the flow path of the conventional AutovaxID™ instrument 4.

FIGS. 4A-4C show an embodiment of the perfusion bioreactor 2 of the invention with a pleated matrix 8, supports 22, and header 24 configured for media distribution. FIG. 4A shows the bioreactor 2 with both body parts 6A, 6B together. FIG. 4B is an exploded view, showing the bioreactor 2 with both body parts 6A, 6B separated and the pleated matrix 8 in between. FIG. 4C shows one body part 6B.

FIGS. 5A-5E an embodiment of the perfusion bioreactor 2 of the invention, having a pleated sheet configuration, including a modified fluid distribution arrangement (referred to herein as the modified pleated sheet configuration). FIGS. 5A and 5E are perspectives of one of the body parts 6B of the bioreactor 2. FIG. 5B is a cross section of both body parts 6A, 6B together, showing direction of inoculation flow. FIG. 5D is a cross section of a body part 6B along line A-A in FIG. 5C. As shown in FIGS. 5A and 5D, in this embodiment, at least one end of the spacers can abut, and be in fluid communication, at least one row of the headers, permitting flow of media there through.

FIGS. 6A-6D show an embodiment of the perfusion bioreactor 2 of the invention, having a spiral wound configuration (referred to herein as the spiral wound bioreactor). FIG. 6A shows the bioreactor 2, including the header 38 and core header 32, with the areas of axial flow (outside) and radial flow (inside) indicted. FIG. 6B shows a cross section of the core 30, membrane 8, and support mesh 34. FIG. 6C shows the core header 32, with membrane 8 (top and bottom) bonded into an envelope with the support mesh 34. The membrane 8 and support mesh 34 are bonded to the core header 32. Preferably, the core has pores 36 for flow of media.

FIGS. 7A-7B show aspects of the conventional AutovaxID™ instrument 4. FIG. 7A is a drawing showing the AutovaxID™ instrument 4, with a hollow fiber bioreactor 40. In The hollow fiber bioreactor typically measures about 8 inches in length and sits inside the disposable cultureware module, enclosed by clear plastic. The fully self-contained system typically contains 4 pumps, a gas exchange cartridge, sensors for pH, temperature, media perfusion and CO₂, heater, and a refrigerated compartment for the storage of media and harvest materials. The system can be equipped to monitor and control lactate levels. FIG. 7B shows a schematic diagram of the AutovaxID™ instrument flow path. In the automated cell culture system of the invention, the system comprises a perfusion bioreactor 2 of the invention in place of a hollow fiber bioreactor 40.

### DETAILED DISCLOSURE OF THE INVENTION

The present invention provides bioreactors 2, automated cell culture systems, and methods for production of cells and cell-derived products. Cells grown using the bioreactors of the invention can be used to rebuild damaged tissue or organs. Potential applications following trauma or injury are numerous, such as for the production of autologous skin for burn repair, growth of bone for fracture repair, and/or the production of tissue for plastic reconstruction of severe injuries.

A plurality of bioreactors 2 of the invention can be run in parallel from a single media source or multiple sources. In one embodiment, three or more bioreactor units 2 are run in parallel from a single media source.

In some embodiments, each bioreactor 2 has a chamber 10, 12 grooved into the housing 6 to hold the cell substrate material 8 (referred to herein as the “matrix”, “cell matrix”, “cell growth matrix”, or “cell substrate matrix”). Any porous material capable of supporting growth of the desired cell type or cell types can be used. In some embodiments, the matrix 8 is constructed of a non-woven polyethylene terephthalate (PET) fabric, which has previously been shown to support 3-D cell interaction and growth of undifferentiated human mesenchymal stem cells (hMSCs) (see, for example, U.S. Pat. Nos. 6,875,605; 6,943,008; and 7,122,371; Grayson et al., J. Cell Physiol., 2006, 207:331-339; Zhao et al., Biotechnol. Prog., 2005, 21:1269-1280; Li et al., Biomaterials, 2001, 22:609-619, which are each incorporated herein by reference in their entirety).

Commercially available PET matrix 8, with a thickness of 1.2 mm, can be treated with NaOH at high temperature, then thermally compressed and cut into sheets. Each chamber 10, 12 is grooved to hold one sheet of PET matrix 8 within a polycarbonate housing 6. The flow chambers 10, 12 are designed such that media will be directed to flow either in parallel to the matrix 8 during cultivation or tangential to the matrix 8 during cell seeding and harvesting. This design integrates cell seeding, long-term cultivation, and harvesting from the matrix 8 of each individual perfusion chamber 2 to facilitate system automation and ease of operation. The capability to modulate media flow in the perfusion chambers 2 also allows for maximum control of the cell growth environment inside the 3-D matrices to meet the varying demands of cell (e.g., hMSC) growth over an extended period to produce large quantities of desired cell populations.

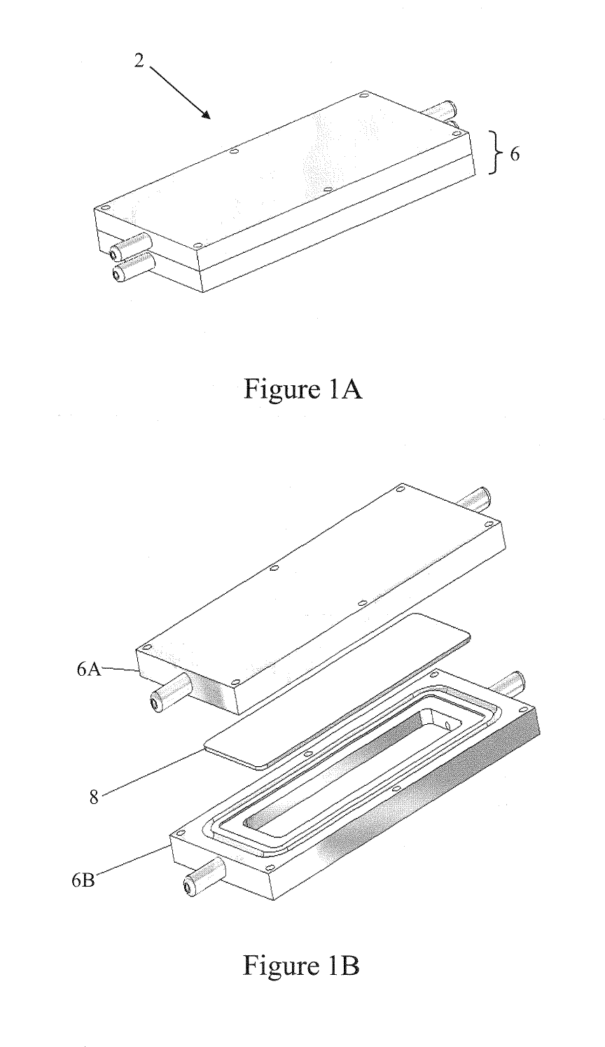

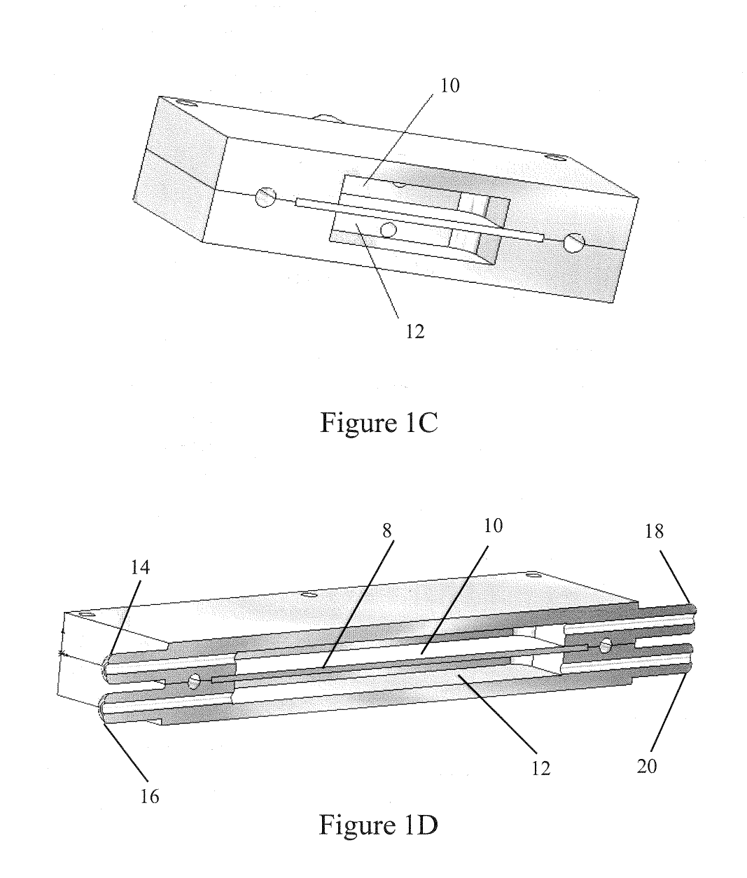

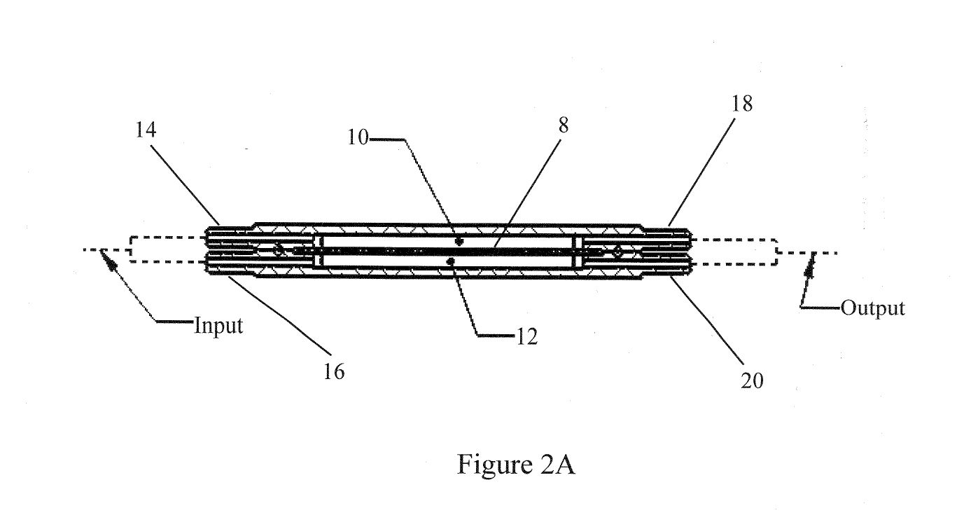

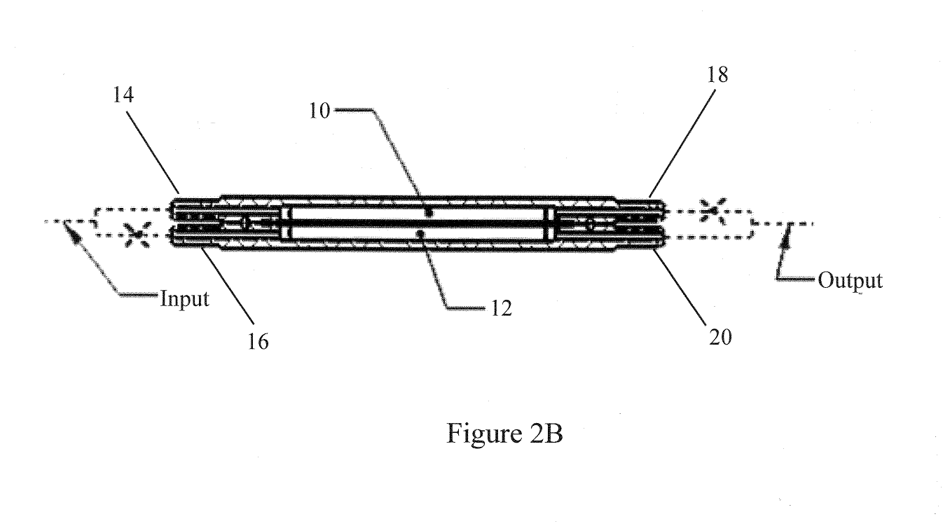

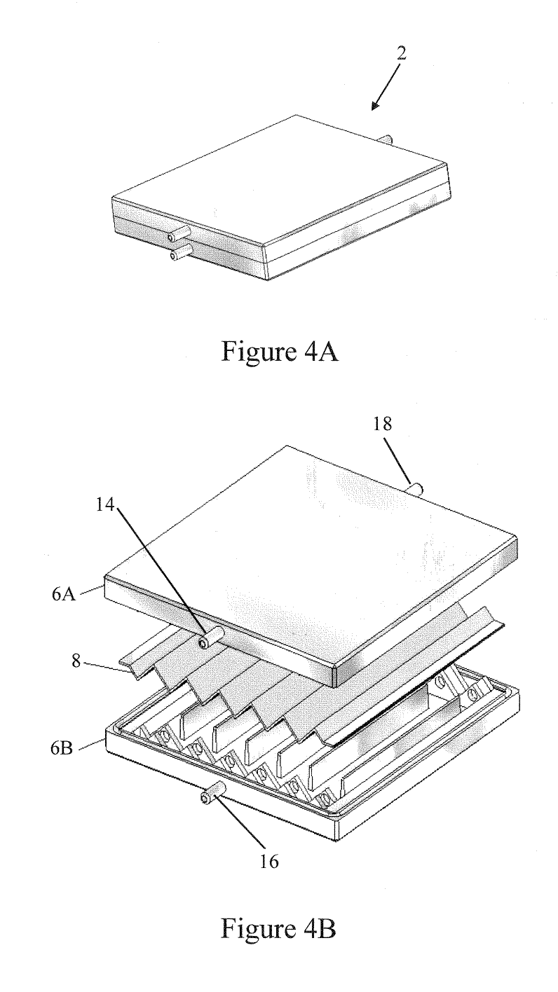

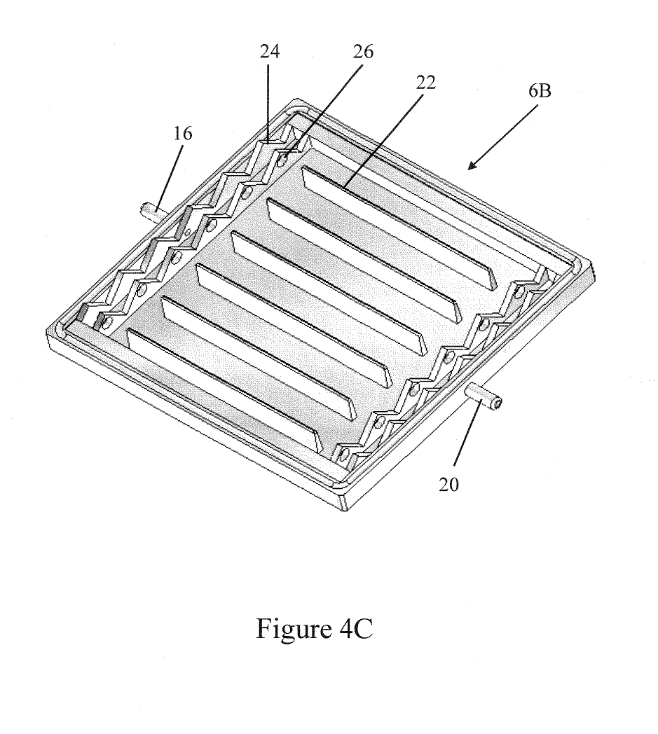

Referring to FIGS. 1A-1D, the main body of each individual bioreactor unit 2 can be machined from medical grade polycarbonate or other suitable material. Preferably, the bioreactor 2 is configured with a first and second part (e.g., top and bottom parts) 6A, 6B, which are designed to removably engage with each other in a fluid-tight manner (e.g., with O-rings or other gaskets), and are preferably symmetrical. The two parts 6A, 6B can be fastened together and sealed to hold the cell growth matrix 8 (shown in FIG. 4) and create a space that is at least partly occupied by the matrix 8, and thereby creating a chamber 10, 12 on either side of the matrix 8 (e.g., above and below the matrix, depending upon the orientation of the bioreactor 2 and matrix 8). Each chamber further comprises an inlet port and outlet port for fluid input/output (e.g., at the end of each chamber).

If PET is to be used as the matrix 8, the PET material can be treated as described in Li et al., Biomaterials, 22:609-618 (2001). Commercial needle punched nonwoven PET fabric, also known as Dacron (fiber diameter: ˜20 μm fiber density: 1.35 g/cm³) is first washed and then hydrolyzed with 1% NaOH solution at 100° C. for 1 hour to reduce surface hydrophobicity. The fabric is then thermally compressed at 120° C. under a pressure of 30 kPa for 45 minutes. The porosity of the matrix should be about 89% with pore size ranging from 20 to 50 μm. Finally, the matrix is cut to fit within the bioreactor body. Each bioreactor will be 2.5 cm×10 cm., have a surface area of 25 cm² and a total matrix volume of ˜3 mL (assuming a matrix thickness of 0.12 cm).

In some embodiments, the bioreactor 2 has a pleated cell growth matrix 8. The main body 6 of the bioreactor 2 can be injection molded from medical grade polycarbonate for production quantities, for example. The unit can be configured with first and second parts 6A, 6B (e.g., top and bottom parts). The two parts will be removably fastened together and sealed to contain a cell growth matrix as shown in FIGS. 4A-4C. This will create a chamber 10, 12 on each side of the matrix 8 (e.g., above and below the matrix 8, depending upon orientation). On the end of each chamber 10, 12, a port exists to allow for fluid input/output (14, 16, 18, 20). Internally, the bioreactor 2 will have a fluidic header space to allow for equal flow distribution along each pleat of the matrix 8.

To construct the pleated design, matrix material can be formed into a pleated surface using concentric rollers. The pleated membrane will then be cut and the side formed to substantially conform to the sealing surface of the bioreactor 2. The membrane will preferably be roll formed, rather than stamped, to maintain the integrity of the pore structure. In one embodiment, the approximate bioreactor available surface area will be 19.1 cm (effective width of pleats)×13.1 long, or 251 cm². The volume of the matrix 8 will be dependant on the final configuration and thickness of the matrix material, but for the typical 1.2 mm thick PET material, this will create a matrix volume of ˜30 mL. Other materials can be used as a cell support matrix.

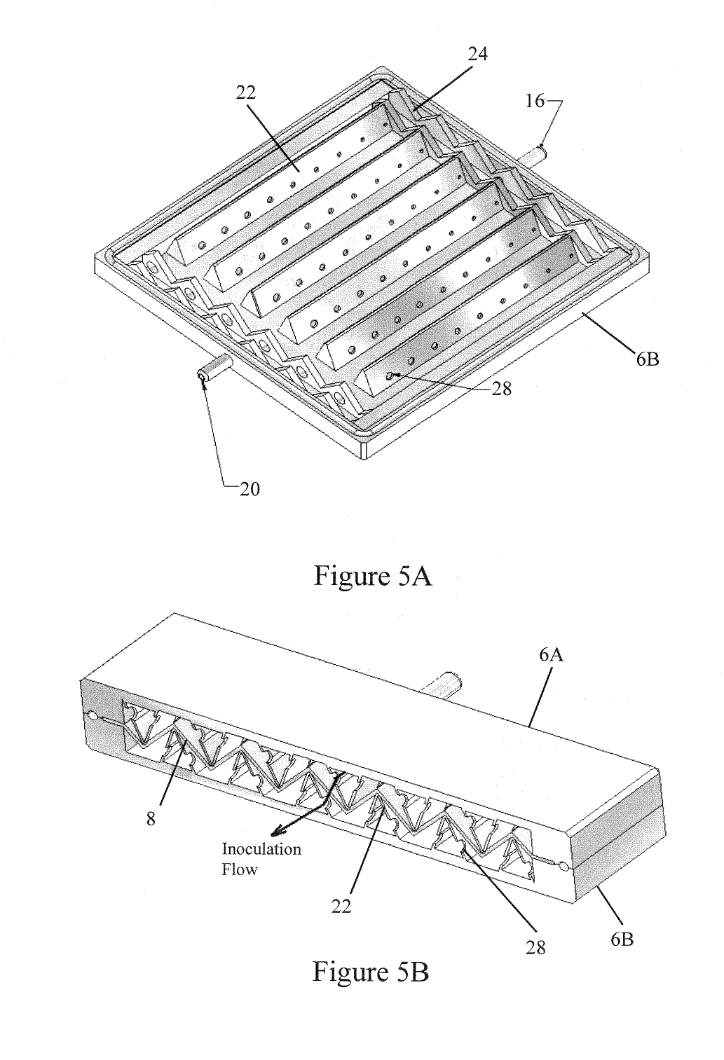

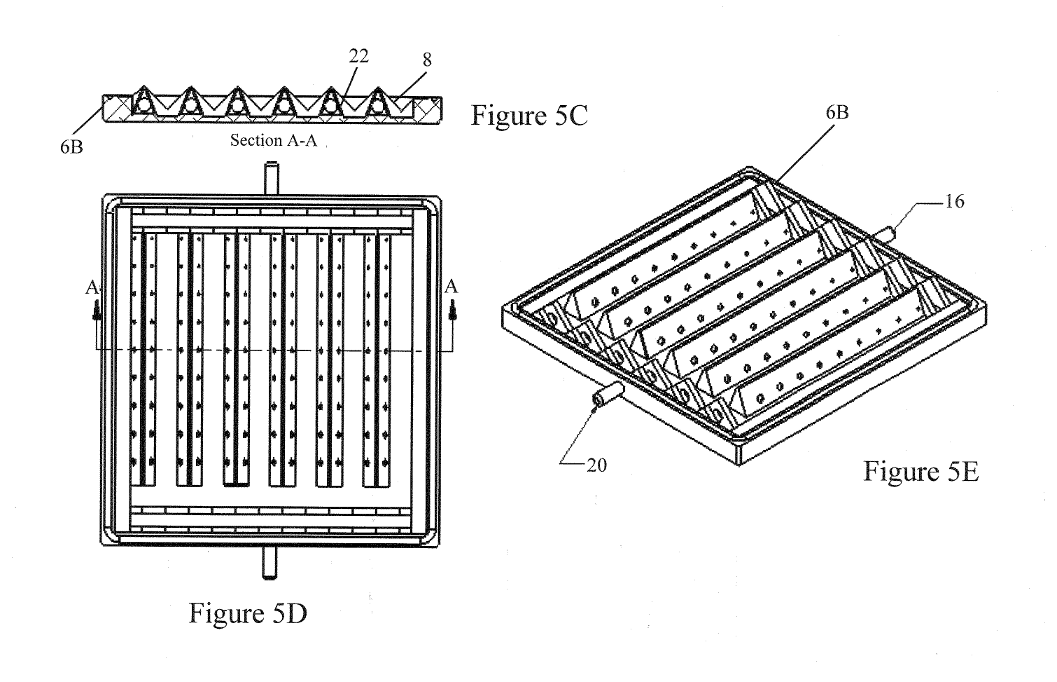

Uniform flow and thorough distribution of cells during inoculation is important for successful large scale cell production. Flow dynamics are also important to minimize gradient formation and evenly deliver nutrients throughout the entire matrix volume. To maximize fluid distribution, an alternate fluid distribution scheme can be used, as shown in FIGS. 5A-5E. Essentially, the bioreactor support structure 22 for the matrix 8 is altered to make it tubular. The fluid enters the support tube from the inlet header and be exposed to the chamber space via a series of holes 28 (preferably, increasing in diameter) along the length of the tube. This embodiment has the potential to improve media distribution and reduce inoculation, nutrient, and oxygen gradients from the chamber inlet 16 to the chamber outlet 20.

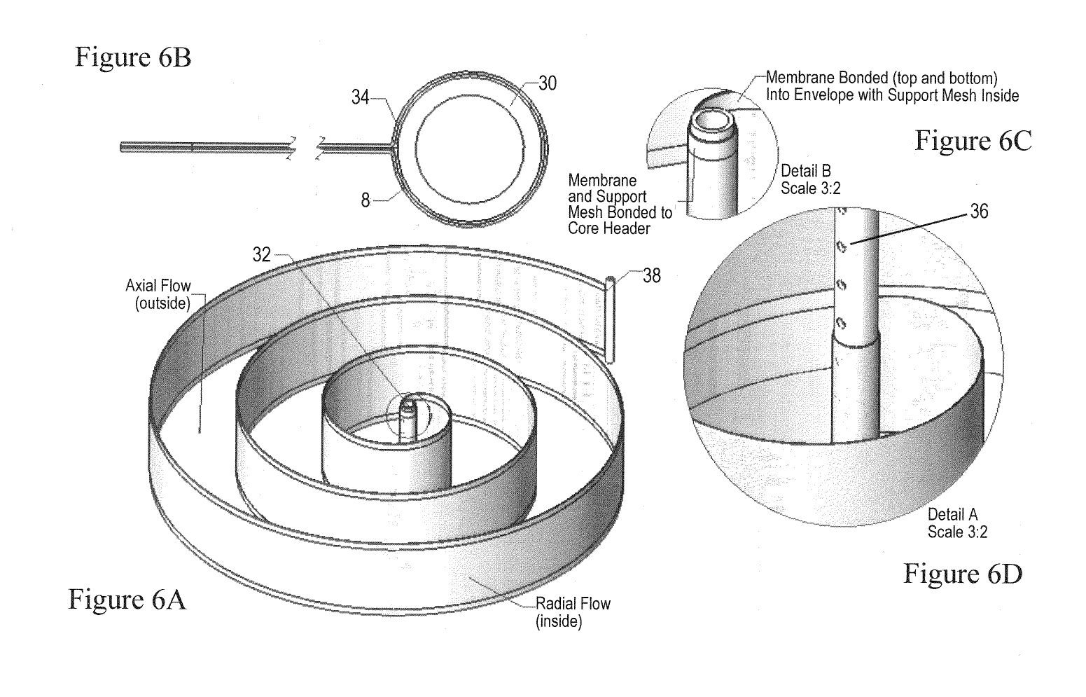

For some therapeutic applications, a bioreactor capable of generating 10⁹ to 10¹¹ cells is desirable. Based on projected cell growth densities for anchorage-dependent lines, this translates into a very large surface area, or matrix volume, that must be contained within a single bioreactor. One way to accomplish this is to spirally wind the matrix 8 around a center core 30, resembling the concept used in tangential flow filtration (TFF) cartridge designs. This technology can be adapted to a bioreactor design that contains a large total volume of the cell substrate matrix 8. Media flow could be directed through separate compartments on either side of the cell substrate matrix 8. This approach involves spirally winding an envelope with a sealed matrix 8 on each side of a porous support mesh 34. The envelope is configured with a fluidic header at each end 32, 38 that acts as the input and output of the “inner envelope chamber”. The envelope will be spiral wound along with an additional support mesh to form the “outer envelope chamber”. This assembly is placed in an enclosure (body 6) with ports (inlet and outlet) on each end. Axial flow is allowed along the outer envelope chamber. Additional enclosure ports are attached to each end of the inner envelope to allow for radial fluid flow. Schematic diagrams of this embodiment are shown in FIGS. 6A-6D. This spiral embodiment is particularly suited for large scale application of allogeneic cells, or other specialized anchorage dependent cells.

Biovest International, Inc. has over 20 years of experience in the area of hollow fiber perfusion bioreactors. The company has recently introduced a new generation cell culture instrument, the AutovaxID™, which is a self-contained, automated system for research, biotechnology and pharmaceutical applications. The AutovaxID™ system includes a base, or control module, which contains the mechanics and electronics, and a disposable cultureware module which is a single-use element containing a hollow-fiber cell growth chamber and a gas exchange cartridge. The cultureware unit is an integrated, sealed module that snaps easily into the base unit housing. Once the inoculum cells are introduced into the unit through an access port, the unit is sealed and the cells expand and grow in a temperature and CO₂-regulated environment, optimized for their specific needs. Nutrients and O₂ are delivered and waste products removed through the automated perfusion of media. Separate pumps control the introduction of growth supplements and the harvest of cell conditioned media. The system can be maintained with continuous cell growth for days to months with little or no operator intervention. All operational parameters during the course of a run are recorded, producing an electronic batch record for documentation and batch-to-batch reproducibility. The use of multiple AutovaxIDs allows of the simultaneous culture of different products in a single facility with minimal environmental control.

The AutovaxID™ cultureware module is a pre-sterilized unit incorporating a hollow fiber perfusion bioreactor. Cells grow on the outside or extracapillary (EC) space while media is perfused through the lumen or intracapillary (IC) space of the hollow fibers. Nutrients and oxygen diffuse into the EC space and waste products diffuse out for their removal. The hollow fiber membrane has a cutoff of 10 KDa, so large molecular weight species such as serum components, growth factors and cell secreted products are confined to the EC compartment. Cells can achieve extremely high densities in the EC compartment due to the efficient perfusion of nutrients. Secreted products, such as monoclonal antibodies, are collected in a relatively small volume at high concentration which aids downstream processing. Culture conditions are highly controlled and consistent throughout a production run and product can be collected at predefined intervals in an automated fashion.

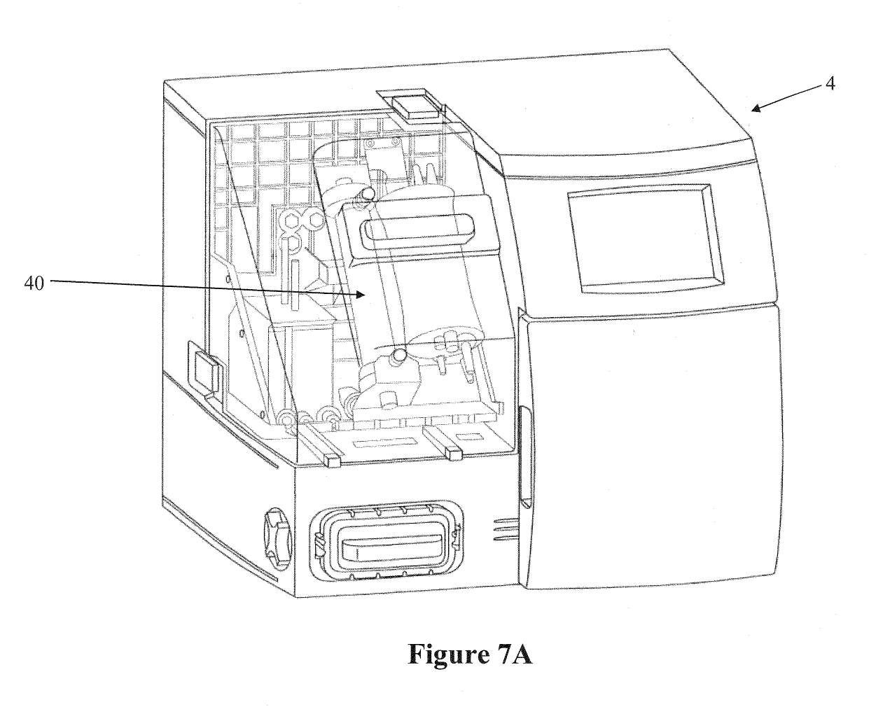

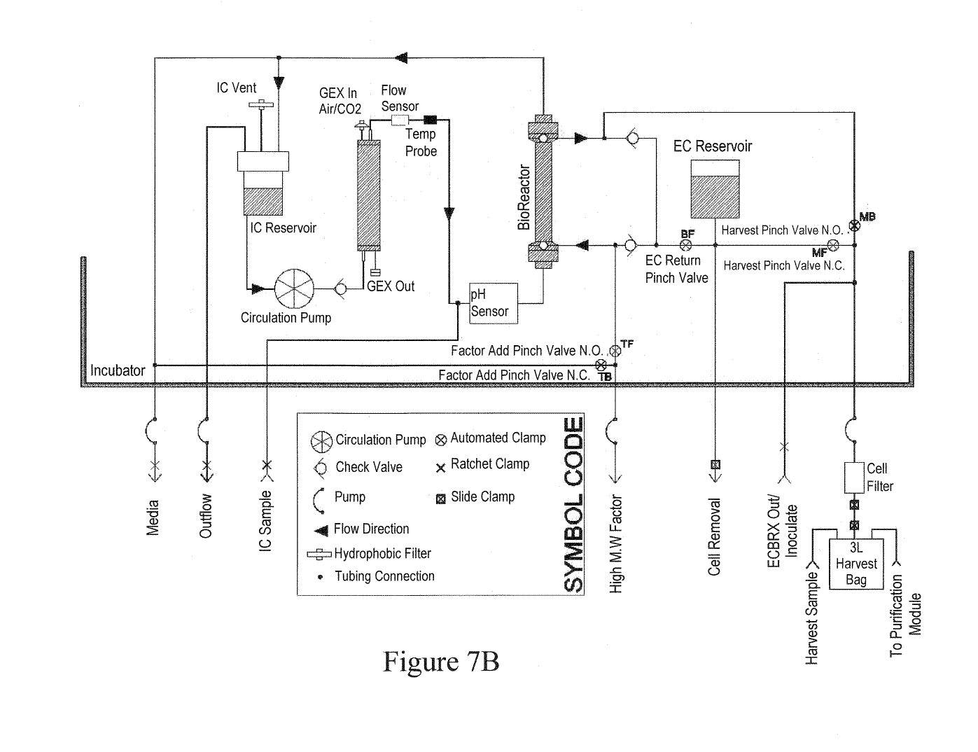

The AutovaxID™ system offers a number of key advantages for the production of stem cell and other cell types. It is simple to use, self contained and can function on the benchtop, eliminating the need for incubators and costly environmental controls. Cells can achieve high density under uniform, controlled and reproducible culture conditions. Automated control means fewer manipulations which minimizes the risk of errors and contamination. Significant savings will be realized in both labor and infrastructure costs. A drawing of the AutovaxID™ instrument and a schematic of the instrument flow path are shown in FIGS. 7A and 7B, respectively.

One or more perfusion bioreactors of the invention can be integrated into the automated cell culture systems and methods described in International Publication No. WO 2007/139742 (Wojciechowski et al.; Method and System for the Production of Cells and Cell Products and Applications Thereof), filed May 21, 2007, and International Publication No. WO 2007/139747 (Page; Interface of a Cultureware Module in a Cell Culture System and Installation Method Thereof), filed May 21, 2007, which are each incorporated by reference herein in their entirety, and referred to as AutovaxID™. In general, the AutovaxID™ is a platform device that automates mammalian cell culture. By incorporating additional bioreactor configurations, the technology can be applied to many specialized cell types or applications. More importantly, complex protocols, normally associated with skilled technicians, can be adapted to perfusion culture with feedback control for reproducible, efficient mass production. By creating a versatile platform technology, the AutovaxID™ system represents a significant advantage over conventional cell culture methods. In general, the ability to simultaneously expand hundreds of autologous (individual patient) cell lines in one facility will play an important role in the commercial application of skin graft and other cell therapies.

A perfusion bioreactor, such as a membrane-based bioreactor, can be used that facilitates expansion and collection of any desired cell type or types. The process control scheme and associated bioreactor for these specialized cell types can be incorporated into the platform AutovaxID™ system. The bioreactor(s) may be, for example, a 3D matrix, flat sheet, pleated sheet, or spiral wound configuration. Preferably, the bioreactor is accessible, or easily removed from the AutovaxID™ system to recover the cells or tissue (e.g., skin cell layer). The systems of the invention incorporate the advantages of automated perfusion methodology to facilitate cost-effective (minimal technician time) production of therapeutic cells. The process control scheme and associated bioreactor for this specialized application can be incorporated into the platform AutovaxID™ system.

Thus, the present invention provides a fully integrated system for producing cells and cell-derived products in a closed, self-sufficient environment. More specifically, the system allows for cell expansion and harvest of cells and their products with minimal need for technician interaction. As will be described further herein, the device incorporates bioreactor perfusion technology, with all tubing components, harvest tubing and tubes threaded through the pump cassette, encased in a single-use, disposable incubator. Following bioreactor inoculation with cells, the system follows pre-programmed processes to deliver media, maintain pH, maintain lactate levels, control temperature and harvest cells or cell-secreted protein. Standard or unique cell culture growth parameters can be programmed, such that, various cell types can be expanded and such that cells or cell products can be harvested in an efficient, reproducible manner with minimal chance of human error.

Medium is perfused through one or more bioreactors. The medium can be a liquid containing a well defined mixture of salts, amino acids, and vitamins that often contain one or more protein growth factors. This serves to deliver nutrients to the cell space and conversely, removes or prevents a toxic build-up of metabolic waste. During this circulation, medium is passed through an oxygenator or gas exchanger cartridge which serves to provide pH control and oxygen for the cells and conversely, remove carbon dioxide from the culture. When the bioreactor contains a smaller number of cells, just after inoculation, the oxygenator or gas exchange cartridge is used to provide CO₂ and subsequently control pH of the culture environment. As cell number increases, the oxygenator is used to remove CO₂ which serves to enhance acid neutralization and control the pH of the culture.

A wide variety of media, salts, media supplements, and products for media formulation can be utilized to produce the cells, depending upon the particular cell type or types. Examples of these substances include, but are not limited to, carrier and transport proteins (e.g., albumin), biological detergents (e.g., to protect cells from shear forces and mechanical injury), biological buffers, growth factors, hormones, hydrosylates, lipids (e.g., cholesterol), lipid carriers, essential and non-essential amino acids, vitamins, sera (e.g., bovine, equine, human, chicken, goat, porcine, rabbit, sheep), serum replacements, antibiotics, antimycotics, and attachment factors. These substances can be present in various classic and/or commercially available media, which can also be utilized with the subject invention. Examples of such media include, but are not limited to, Ames' Medium; Basal Medium Eagle (BME), Click's Medium, Dulbecco's Modified Eagle's Medium (DMEM), DMEM/Nutrient Mixture F12 Ham, Fischer's Medium, Minimum Essential Medium Eagle (MEM), Nutrient Mixtures (Ham's), Waymouth Medium, and William's Medium E.

The system provides significant efficiencies and cost reduction through its disposable component and enclosed operation. As such, cells are contained in a closed system and continuously cultured without the need for specialized, segregated clean rooms. This fully integrated apparatus eliminates the need for cleaning and sterilization validations, as well as the need for hard plumbing associated with conventional cell culture facilities.

The system includes two individual parts: an instrumentation base device that is reusable and an enclosed cultureware module that is used for a single production run and is disposable, which is particularly advantageous for therapeutic applications. Numerous modules can be used on a single device. The instrument provides the hardware to support cell culture growth and production in a compact package. An easy-load multiple channel peristaltic pump drive located in the base device and a pump cassette move fresh basal media into the cultureware, removes spent media, adds growth factors or other supplements and removes product harvest. An integrated cool storage area maintains the factor and harvest at a low temperature (approximately 4° C.). An integrated heating mechanism maintains the cell environment to promote growth and production. Gas exchange cartridge, in conjunction with a cultureware pH sensor controls the pH of the cell culture medium. Two automated tube valving drives can be used to control the cultureware flow path configuration to accomplish the fluidic switching functions needed to initiate and do a successful run. Valves and sensors in the instrument control the fluid cycling in the cultureware module. A pump drive for fluid circulation is provided. An attached barcode reader can be used to facilitate operator and lot tracing. A communication port ties the instrument to a data information management system (such as a MES). A flat panel display with touch screen can be incorporated for user interaction.

The one-time use cultureware module is provided pre-sterilized. It is designed for quick loading onto the instrument (“quick-load”). The loading of the cultureware body makes connections to the instrument. The pump cassette, which is physically attached to the tubing, allows the user to quickly load the pump segments. This design and layout minimizes loading errors. The cultureware enclosure provides an area that is heated to maintain cell fluid temperature. A fluid cycling unit maintains fluid volumes and cycling and is included in the cultureware. Sensors for fluid circulation rate, pH and a thermal well for the instrument's temperature sensor are provided. The blended gas from the instrument is routed to gas exchange cartridge that provides oxygen and adds or removes carbon dioxide to the circulated fluid to support cell metabolism. A magnetically coupled pump drive circulates fluid thru the bioreactor and gas exchange cartridge. At least one bioreactor, which provides the cell space and media component exchange is also in the cultureware. Disposable containers for harvest collection are provided. Prior to the beginning of the culture, the operator attaches a media source, factor bag and spent media container to the cultureware before running. At the conclusion of the run, the harvest containers are removed or drained, media and spent media container is disconnected, pump cassette is unloaded, harvest bag disconnected, cultureware body is unloaded and the used cultureware is placed in a biohazard container for disposal.

Cell expansion and subsequent process tracking necessitates generation of a batch record for each culture. Historically this is done with a paper-based system that relies on operator input of the information. This is labor intensive and subject to errors. The fully integrated device can incorporate a barcode reader and data gathering software which, when used with the information management system (MES), allows for automatic generation of the batch record.

The system of the present invention has application in a regulated cell culture environment. It is anticipated that autologous whole cell therapies or patient-specific proteins (vaccines) therapies, would by their nature, require the simultaneous culture of numerous cell lines in a single facility. In addition to the segregation created through this closed culture approach, the apparatus is designed to support a standard information management system (such as a LIMS or MES) protocol. This capability contributes to the creation of thorough batch records and verification of culture conditions to ensure standardization, tracking and safety of each product. This capability facilitates the multi-product concept that is pivotal to facilities involved with autologous or patient-specific products.

The disposable cell culture module is removably attachable to the device. The module requires multiple mechanical and electrical interfaces to the control instrumentation of device. The module has interface features integrated into the module that mate with instrument interface features in the device to allow for a single motion installation. As the modules are to be disposed of after use, it should be appreciated that numerous modules can be used in conjunction with a single base device.

The interface features of the device include the circulation pump drive, actuator valves and cycling sensor. In addition, a temperature probe and a flow sensor interface with the components of the module. The device also includes an electrical connection for the pH probe disposed within the module.

Gas ports communicate with a gas exchanger. One port communicates with the input to the exchanger and the other port communicates with the output of the exchanger. Gas ports control pressure to the cycling fixture.

As described above, the module is heated to maintain cell fluid temperature. The heating mechanism maintains the cell environment to promote growth and production. The cell culture, disposable modules requiring elevated temperatures are warmed by fully encapsulating the module and attaching the module to the controlling instrument, such that air ports are aligned and warmed air is forced into the module from the instrument at one location and allowed to escape at another. The instrument device has a heated air outlet and a return heated air inlet.

Referring to the flow diagram of the conventional AutovaxID system in FIG. 7B, the pump moves fresh basal media into the cultureware at the media line. The media line is connected to a user provided container of fresh media to provide the growth nutrients to the cell culture that are pumped into the disposable. The outflow line is connected to a user provided container to collect the waste or spent media being pumped out of the disposable. The factor line is connected to a user provided container of growth factors that are pumped into the disposable. EC inoculate and IC sample can be added where indicated. The product harvest is removed as indicated. The cells are harvested as indicated. The harvest line is a pre-attached container that is part of the disposable that is used to collect the product that is pumped out of the disposable. The pump has multiple lines. Because the pump has a common fixed axial shaft and individual servo driven rotors, the control of the flow of each can be independent, allowing one channel or flow to be increased while another decreased.

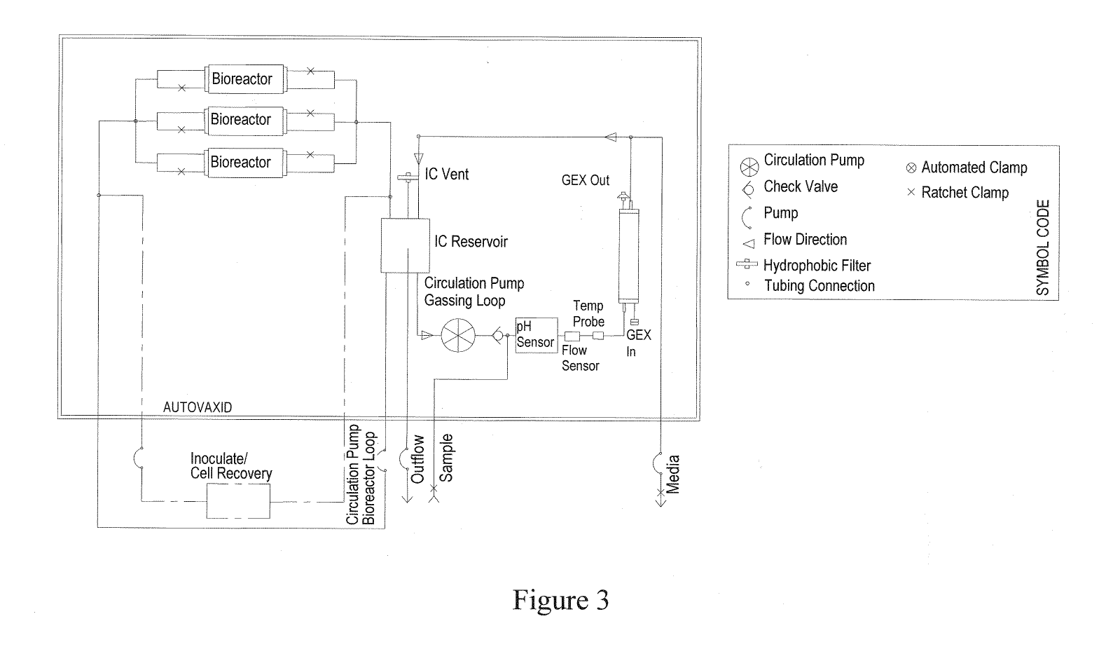

The AutovaxID™ instrument has been designed to control the pumps, gas exchange, and sensors in either automatic mode or manual override condition. These modes are selectable by the operator. Manual control will allow for experimentation with selected variables to maximize operation and flexibility of the system. The cultureware module will have significant changes to allow for the anticipated low flow rate required for inoculation and recirculation. System flow rates in the range of 0.1 mL/min are anticipated for the operation of the bioreactor. Two fluidic paths will be incorporated into the cultureware: a recirculation/inoculation loop and an oxygenation loop. Both circuits will feed from the same reservoir as shown in FIG. 3.

The oxygenation circuit consists of a centrifugal pump (e.g., 500 mL/minute maximum) that takes media from the reservoir and pumps it through a gas exchange cartridge (GEX) and back to the reservoir. The circuit also houses sensors that monitor flow rates (volume) and pH of the media. The AutovaxID control system uses the measured pH reading to adjust the gas blend delivered to the GEX, thereby oxygenating the media and adjusting the pH to maintain a user-defined set point. This is accomplished by providing a gas blend of air/CO₂ which diffuses through the GEX membrane and equilibrates with the bicarbonate buffered media. The GEX also acts to remove unwanted media waste gas (e.g., ammonia, excess CO₂). The reservoir is continually fed with fresh media to replenish nutrients and maintain the reservoir volume that is continually being reduced by the outflow/waste removal pump (media feed rate plus 20%). The end result is a controlled level of oxygenated, pH adjusted media in the reservoir.

The recirculation circuit uses a peristaltic pump (0 to 400 mL/hour or 0 to 6.67 mL/min) to deliver oxygenated and pH controlled media from the reservoir, through the bioreactor, and back to the reservoir. Another peristaltic pump (0 to 400 mL/hour) drives an additional feed line (growth supplement or factor addition) which is connected to this circuit. This feed line allows for the introduction of supplementary materials such as growth factors and other media components to the main primary media circuit.

The oxygenation and the recirculation circuits will be enclosed within a clear polycarbonate shell that also serves as a temperature incubator. The AutovaxID forces controlled-temperature air into the enclosure to regulate the temperature of the media and bioreactor. The cultureware will be pre-assembled and ethylene oxide sterilized. Media, factor and waste containers are aseptically connected by the operator.

The bioreactors of the invention will replace the hollow fiber bioreactor traditionally housed in the AutovaxID flowpath. Briefly, each flowpath may contain one or more bioreactors under process control of a single AutovaxID control unit. These bioreactors will preferably be positioned parallel to each other and share a common media feed. It is possible that at low flow rates, media may not distribute evenly to all three bioreactors. This will be evaluated by changing media composition (osmolality, or protein concentration) and monitoring output from each bioreactor during engineering development runs. If the flow is not uniform to all bioreactors, additional efforts to manifold media flow will be conducted by the engineering group. If media cannot be delivered simultaneously to all three bioreactors, then two bioreactors will be removed and subsequent flowpaths will be assembled with only one bioreactor.

Under the current AutovaxID operating conditions for the growth of hybridoma lines, cells are maintained in the space surrounding the hollow fibers, while media is continuously perfused through the lumen of the fibers. To enhance nutrient delivery and metabolic waste removal, a pressure differential is created across the cell-side and non-cell side of the fiber membrane. This pressure differential is then reversed every 15 minutes to remove waste and evenly distribute nutrients throughout the densely packed cell population. This key capability may be adapted for the Phase I prototype bioreactor with the 3-D cell growth matrix to enhance the cell seeding process. By creating a pressure differential, cells may penetrate more effectively and distribute more evenly in the PET matrix. Prior studies have shown significant improvement of cell seeding efficiency and distribution in the 3-D matrices by employing the dynamic seeding methods. In addition, the capability to direct the cell harvesting solutions to penetrate the 3-D matrix may greatly enhance the efficiency of cell recovery using automated procedures. It should be noted that the shear stress applied during cell seeding and harvesting is low (<<1 dyn/cm²) and the duration of operation is short (<1 hr). In fact, the dynamic application of cell dissociation buffer to the cells embedded in the 3-D matrix at low flow rate is expected to reduce exposure of the cells to the enzymatic solution and limit any cellular damage.

The cells grown using the invention can range in plasticity from totipotent or pluripotent stem cells (e.g., adult or embryonic), precursor or progenitor cells, to highly specialized cells, such as those of the central nervous system (e.g., neurons and glia). In some embodiments, the cells are bone marrow cells, hematopoietic stem cells or hematopoietic progenitor cells, mesenchymal stem cells, or other stem cells or progenitor cells. The cells may be administered to a subject in an enriched (e.g., purified or isolated) or non-enriched form. Stem and progenitor cells can be obtained from a variety of sources, including embryonic tissue, fetal tissue, adult tissue, umbilical cord blood, peripheral blood, bone marrow, and brain, for example.

In some embodiments, the cells are human cells. However, it will be understood by one of skill in the art that the present invention is also applicable for veterinary purposes. Cells of non-human animals can find application either in human or animal subjects (transplant recipients). For example, although dopamine neurons from human, pig, and rat are similar in that they synthesize dopamine and release synaptically into the brain, they differ immunologically, in extent of reinervation of the brain, in life span, and in infection agents associated with the specific donor or donor species. These traits can be exploited for their specific strengths and weaknesses.

As will be understood by those skilled in the art, there are over 200 cell types in the human body. The bioreactors, systems, and methods of the subject invention can be used to grow any of these cell types. For example, cells can include those cells arising from the ectoderm, mesoderm, or endoderm germ cell layers. Such cells include, but are not limited to, bone marrow cells, neurons, glial cells (astrocytes and oligodendrocytes), muscle cells (e.g., cardiac, skeletal), chondrocytes, fibroblasts, melanocytes, Langerhans cells, keratinocytes, endothelial cells, epithelial cells, pigment cells (e.g., melanocytes, retinal pigment epithelial (RPE) cells, iris pigment epithelial (IPE) cells), hepatocytes, microvascular cells, pericytes (Rouget cells), blood cells (e.g., erythrocytes), cells of the immune system (e.g., B and T lymphocytes, plasma cells, macrophages/monocytes, dendritic cells, neutrophils, eosinophils, mast cells), thyroid cells, parathyroid cells, pituitary cells, pancreatic cells (e.g., insulin-producing beta cells, glucagon-producing alpha cells, somatostatin-producing delta cells, pancreatic polypeptide-producing cells, pancreatic ductal cells), stromal cells, adipocytes, reticular cells, rod cells, and hair cells. Other examples of cell types that can be grown include those disclosed by Spier R. E. et al., eds., (2000) The Encyclopedia of Cell Technology, John Wiley & Sons, Inc., and Alberts B. et al., eds., (1994) Molecular Biology of the Cell, 3^{rd} ed., Garland Publishing, Inc., e.g., pages 1188-1189.

Various cell lines have also been used for a variety of purposes, and can be grown using the bioreactors, systems, and methods of the invention. Fetal kidney cells and amniotic cells have been transplanted as sources of trophic factors. Adrenal medullary cells, sympathetic ganglion cells, and carotid body cells have been transplanted as sources of dopamine. Fibroblasts and glial cells have been transplanted as sources of trophic factors, to carry genes through recombinant strategies, or for demyelinating diseases, for example. Corneal endothelial cells have been used for corneal transplants. Myoblasts have been transplanted for the treatment of muscular dystrophy and cardiac disease. Other cell lines include pancreatic islet cells for diabetes; thyroid cells for thyroid disorders; blood cells for AIDS, bone marrow transplant, and inherited disorders; bone and cartilage for osteoarthritis, rheumatoid arthritis, or for fracture repair; skin or fat cells for reconstructive purposes, such as in skin grafts after burns or cosmetic surgery; breast augmentation with fat; hair follicle replacement; liver cells for liver disorders inducing hepatitis; and retinal pigment epithelial cells (RPE) for retinitis pigmentosa and Parkinson's disease.

The cells to be used in the various aspects of the present invention are preferably mammalian cells. They may be of human or animal origin. Examples of mammalian cells that can be grown using the bioreactors, systems, and methods of the invention include, but are not limited to, murine C127 cells, 3T3 cells, COS cells, human osteosarcoma cells, MRC-5 cells, BHK cells, VERO cells, CHO (Chinese hamster ovary) cells, HEK 293 cells, rHEK 293 cells, normal human fibroblast cells, Stroma cells, Hepatocytes cells, or PER.C6 cells. Examples of hybridomas that may be cultured in the process according to the present invention include, e.g., DA4.4 cells, 123A cells, 127A cells, GAMMA cells and 67-9-B cells.

Stem cells are believed to have immense potential for therapeutic purposes for numerous diseases. Stem cells have been derived from numerous donor sources, including, but not limited to, embryonic, blast, tissue-derived, blood, and cord-blood cells; organ-derived progenitor cells; and bone marrow stromal cells, among others. Such stem cells can be differentiated along numerous pathways to produce virtually any cell type. These cells can be transplanted either before or after differentiation. Hematopoietic stem cells (HSC) have been used for many years, and typically used for treatment of hematopoietic cancers (e.g., leukemias and lymphomas), non-hematopoietic malignancies (cancers in other organs). Other indications include diseases that involve genetic or acquired bone marrow failure, such as aplastic anemia, thalassemia sickle cell anemia, and autoimmune diseases.

Methods and markers commonly used to identify stem cells and to characterize differentiated cell types are described in the scientific literature (e.g., Stem Cells: Scientific Progress and Future Research Directions, Appendix E1-E5, report prepared by the National Institutes of Health, June, 2001). The list of adult tissues reported to contain stem cells is growing and includes bone marrow, peripheral blood, umbilical cord blood, brain, spinal cord, dental pulp, blood vessels, skeletal muscle, epithelia of the skin and digestive system, cornea, retina, liver, and pancreas.

Cells and cells-derived products can be harvested using methods known in the art. Various biomolecules produced by genetically modified or non-genetically modified cells that are produced using the bioreactors, systems, and methods of the subject invention can be harvested (e.g., isolated from the biomolecule-producing cells) for various uses, such as the production of drugs and for pharmacological studies. Thus, using the bioreactors, systems, and methods of the invention, cells can be used as biological “factories” to provide the product of exogenous DNA and/or the natural product of the cells in vitro, or in vivo within an animal. The term “biomolecule” refers to molecule or molecules that can be produced by cells (a cell-derived product). Such biomolecules include, but are not limited to, proteins, peptides, amino acids, lipids, carbohydrates, nucleic acids, nucleotides, viruses, and other substances. Some specific examples of biomolecules include trophic factors, hormones, and growth factors, such as brain-derived growth factor (BDNF) and glial-derived neurotrophic factor (GDNF). For example, pituitary cells can be grown to produce growth hormone; kidney cells can be grown to produce plasminogen activator; bone cells can be grown to produce bone morphogenetic protein (BMP) or other proteins involved in bony fusions or prosthetic surgery. Hepatitis-A antigen can be produced from liver cells. Cells can be grown to produce various viral vaccines and antibodies. Interferon, insulin, angiogenic factor, fibronectin and numerous other biomolecules can be produced by growing cells and harvesting these products. The biomolecules can be intracellular, transmembrane, or secreted by the cells, for example.

The biomolecule can be a polypeptide of interest, such as a naturally secreted protein, a normally cytoplasmic protein, a normally transmembrane protein, or a human or a humanized antibody. When the protein of interest is a naturally cytoplasmic or a naturally transmembrane protein, the protein has preferably been engineered in order to become soluble and secreted, i.e., by placing a signal peptide in front of it or of a (soluble or extracellular) fragment of it.

The polypeptide of interest may be of any origin. Preferred polypeptides of interest are of human origin, and more preferably, the proteins of interest are therapeutic proteins. Preferably, the protein of interest is selected from a hormone, a cytokine-binding protein, an interferon, a soluble receptor, or an antibody. Therapeutic proteins that may be produced include, for example, chorionic gonadotropin, follicle-stimulating hormone, lutropin-choriogonadotropic hormone, thyroid stimulating hormone, growth hormone, in particular human growth hormone, interferons (e.g., interferon beta-1a, interferon beta-1b), interferon receptors (e.g., interferon gamma receptor), TNF receptors p55 and p75, and soluble versions thereof, TACI receptor and Fe fusion proteins thereof, interleukins (e.g., interleukin-2, interleukin-11), interleukin binding proteins (e.g., interleukin-18 binding protein), anti-CD11a antibodies, erythropoietin, granulocyte colony stimulating factor, granulocyte-macrophage colony-stimulating factor, pituitary peptide hormones, menopausal gonadotropin, insulin-like growth factors (e.g., somatomedin-C), keratinocyte growth factor, glial cell line-derived neurotrophic factor, thrombomodulin, basic fibroblast growth factor, insulin, Factor VIII, somatropin, bone morphogenetic protein-2, platelet-derived growth factor, hirudin, epoietin, recombinant LFA-3/IgG1 fusion protein, glucocerebrosidase, and muteins, fragments, soluble forms, functional derivatives, fusion proteins thereof. In some embodiments, the polypeptide is selected from the group consisting of chorionic gonadotropin (CG), follicle-stimulating hormone (FSH), lutropin-choriogonadotropic hormone (LH), thyroid stimulating hormone (TSH), human growth hormone (hGH), interferons (e.g., interferon beta-1a, interferon beta-1b), interferon receptors (e.g., interferon gamma receptor), TNF receptors p55 and p75, interleukins (e.g., interleukin-2, interleukin-11), interleukin binding proteins (e.g., interleukin-18 binding protein), anti-CD11a antibodies, and muteins, fragments, soluble forms, functional derivatives, fusion proteins thereof.

Further preferred polypeptides of interest include, e.g., erythropoietin, granulocyte colony stimulating factor, granulocyte-macrophage colony-stimulating factor, pituitary peptide hormones, menopausal gonadotropin, insulin-like growth factors (e.g., somatomedin-C), keratinocyte growth factor, glial cell line-derived neurotrophic factor, thrombomodulin, basic fibroblast growth factor, insulin, Factor VIII, somatropin, bone morphogenetic protein-2, platelet-derived growth factor, hirudin, epoietin, recombinant LFA-3/IgG1 fusion protein, glucocerebrosidase, and muteins, fragments, soluble forms, functional derivatives, fusion proteins thereof.

Viruses and viral vectors represent another type of cell-derived product that may be produced using the bioreactors, systems, and methods of the invention. Viruses and viral vectors can be produced with the invention using cell types utilized for propagating the virus of interest. Examples of mammalian cells useful for production of virus include Madin-Darby canine kidney (MDCK) cells, VERO cells, or other monolayer cell types. The cells are grown in the bioreactor of the invention and, after a sufficient cell number is reached, are then infected with the virus or viral vector, which spreads throughout the culture and larger quantities of virus or vector is then harvested. The harvested virus and vectors can be used, for example, for vaccines and/or as gene delivery vectors. For example, influenza virus can be grown and vaccines for influenza produced from the harvested virus. While the roller-bottle and egg-based vaccine production processes remain relatively reliable, an efficient cell-based production system would represent a significant improvement in providing a faster, less-expensive, and less cumbersome method of growing viruses.

The process of manufacturing a viral vaccine comprises the process of replicating a virus using a bioreactor, system, or method of the invention and harvesting the virus, which can include at least one step selected among filtering, concentrating, freezing and stabilizing by addition of a stabilizing agent. The virus harvest can be performed according to technologies well-known to the man skilled in the art. According to a preferred embodiment, the step of harvesting the virus comprises collecting cell culture supernatant obtained from centrifugation, then filtering, concentrating, freezing and stabilizing virus preparation by addition of stabilizing agent. For example, for influenza virus, see Furminger, In Nicholson, Webster and Hay (Eds) Textbook of influenza, chapter 24 pp 324-332.

The process of manufacturing a viral vaccine according to the invention may also comprise the additional step of inactivation of harvested virus. Inactivation can be performed by treatment with formaldehyde, beta-propiolactone, ether, ether and detergent (i.e., such as Tween 80™), cetyl-trimethyl ammonium bromide (CTAB) and Triton N102, sodium deoxycholate and tri(N-butyl)phosphate.

The bioreactors, systems, and methods of the invention may also be used for preparation of viral antigenic proteins from the virus produced therewith. The method further comprises the additional steps of: a) optionally, incubating cell culture supernatant comprising whole virus harvested from the bioreactor with a desoxyribonucleic acid restriction enzyme, preferably DNAses and nucleases (preferably, the DNA digestion enzyme is benzonase (Benzon nuclease) or DNase I); b) adjunction of cationic detergent (examples of cationic detergent are; without limitation: cetyl-trimethyl ammonium salt such as CTAB, myristyl-trimethyl ammonium salt, lipofectine, DOTMA and Tween™); c) isolation of antigenic proteins. This later step may be carried out by centrifugation or ultrafiltration.

The virus in the vaccine may be present either as intact virus particles, or as disintegrated virus particles. According to an embodiment, the vaccine is a killed or inactivated vaccine. According to another embodiment, the vaccine is a live attenuated vaccine. According to a third embodiment, the vaccine comprises viral antigenic proteins obtainable from a virus prepared according to the method of the invention.

The vaccine may comprise the virus in combination with pharmaceutically acceptable substances which increase the immune response. Non-limiting examples of substances which increase the immune response comprises incomplete Freund adjuvant, saponine, aluminium hydroxide salts, lysolecithin, plutonic polyols, polyanions, peptides, bacilli Calmette-Guerin (BCG) and corynebacterium parvum. In addition, immuno-stimulating proteins (e.g., interleukins IL-1, IL-2, IL-3, IL-4, IL-12, IL-13, granulocyte-macrophage-colony-stimulating factor) may be used to enhance the vaccine immune response.

The vaccine is preferably a liquid formulation, a frozen preparation, a dehydrated and frozen preparation, optionally adapted to intra-nasal route of administration.

The vaccine may be used for the prophylactic and/or therapeutic treatment of a human infected by a virus or at risk of infection, or for treatment or prevention of other diseases such as cancer. The viral vaccine may be a recombinant viral vaccine.

Some examples of applications for which the bioreactor, system, and method of the present invention can be used include:
The production of monoclonal antibodies from hybridoma cell lines (e.g., the K6H6/B5 or 1D12 hybridoma cell lines).
The expansion of autologous patient-derived blood cells including immune cells for therapeutic application.
The expansion of patient derived somatic cells for subsequent re-infusion back into patients for therapeutic purposes. A specific example already available for therapeutic application in patients is the harvesting and expansion of patient specific cartilage cells (chondrocytes) followed by re-infusion of those cells back into a region containing damaged articular cartilage.
The expansion of patient derived or generic multipotent cells, including embryonic stem cells, adult stem cells, hematopoeitic stem or progenitor cells, multi- or pluripotent cells derived from cord blood or other sources for therapeutic purposes.
The expansion of somatic or germline cells as in the aforementioned cellular applications and in which the cells have been genetically modified to express cellular components or to confer on them other beneficial properties such as receptors, altered growth characteristics or genetic features, followed by introduction of the cells into a patient for therapeutic benefit. An example is the expansion of patient specific fibroblasts genetically modified to express growth factors, clotting factors, or other biologically active agents to correct inherited or acquired deficiencies of such factors.
The production of virus (such as influenza) and viral vectors, e.g., for production of vaccines.
The production of other cell-derived products such as growth factors.

The specification is most thoroughly understood in light of the teachings of the references cited within the specification which are hereby incorporated by reference. The embodiments within the specification provide an illustration of embodiments in this application and should not be construed to limit its scope. The skilled artisan readily recognizes that many other embodiments are encompassed by this disclosure. All publications and patents cited and sequences identified by accession or database reference numbers in this disclosure are incorporated by reference in their entirety. To the extent that the material incorporated by reference contradicts or is inconsistent with the present specification, the present specification will supersede any such material. The citation of any references herein is not an admission that such references are prior art to the present specification.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

What is claimed is:

1. A perfusion bioreactor, comprising a housing with an inlet port and outlet port, and a cell growth matrix, wherein said cell growth matrix is planar, pleated, or spirally wound around a central core.

2. The perfusion bioreactor of claim 1, wherein said housing comprises a first part with an inner surface and a second part with an inner surface, wherein said first part engages with said second part in a fluid-tight manner and such that said inner surfaces of said first part and said second part define a space occupied by said cell growth matrix, wherein said matrix has a first side and a second side, wherein said first matrix side and said inner surface of said first part define a first chamber, and wherein said second matrix side and said inner surface of said second part define a second chamber.

3. The perfusion bioreactor of claim 2, wherein said first part has an inlet port and an outlet port in fluid communication with said first chamber, and wherein said second part has an inlet port and an outlet port in fluid communication with said second chamber.

4. The perfusion bioreactor of claim 2, wherein said matrix is pleated, and wherein said inner surface of said first part, or said inner surface of said second part, or both, further comprise supports for supporting the pleats of said matrix.

5. The perfusion bioreactor of claim 4, wherein said inner surface of said first part and said inner surface of said second part further comprise headers, wherein said headers of said inner surface of said first part engage with said headers of said inner surface of said second part, and wherein each of said headers has a hole for flow of medium through said header.

6. The perfusion bioreactor of claim 4, wherein each support conforms to each pleat of said matrix.

7. The perfusion bioreactor of claim 4, wherein each support has a hole for flow of medium through said support.

8. The perfusion bioreactor of claim 1, wherein said bioreactor comprises a central core, and said matrix is wound around said central core.

9. The perfusion bioreactor of claim 1, wherein said matrix comprises a material selected from the group consisting of polyethylene terephthalate (PET), collagen, and chitosan.

10. A cell culture system for the production of cells and/or cell-derived products, comprising:
a reusable instrumentation base device incorporating hardware to support a cell culture growth; and
at least one disposable cell cultureware module removably attachable to said instrumentation base device, said module including at least one perfusion bioreactor comprising a housing with an inlet port and outlet port, and a cell growth matrix, wherein said cell growth matrix is planar, pleated, or spirally wound around a central core.

11. The cell culture system of claim 10, wherein said instrumentation device includes a pump for circulating cell culture medium through said at least one cultureware module.

12. The cell culture system of claim 11, wherein said pump moves growth factor or other supplements into the cell growth chamber and removes product harvest from said perfusion bioreactor.

13. The cell culture system of claim 11, wherein said instrumentation device includes a plurality of rotary selection valves to control the medium flow through said at least one cultureware module.

14. The cell culture system of claim 10, wherein said instrumentation device includes a heating mechanism for heating said cell growth chamber to promote growth and production.

15. The cell culture system of claim 11, wherein said at least one cultureware module includes a gas blending mechanism in communication with said cell growth chamber.

16. The cell culture system of claim 10, wherein said bioreactor provides cell space and medium component exchange.

17. A method for the production of cells and/or cell-derived products, comprising the steps of:
providing at least one disposable cultureware module, said module including at least one perfusion bioreactor comprising a housing with an inlet port and outlet port, and a cell growth matrix, wherein said cell growth matrix is planar, pleated, or spirally wound around a central core;
providing a reusable instrumentation base device incorporating hardware to support cell culture growth, said base device including a microprocessor control and a pump for circulating cell culture medium through the bioreactor;
removably attaching said at least one cultureware module to said instrumentation base device;
introducing cells into the bioreactor;
fluidly attaching a source of cell culture medium to said at, least one cultureware module;
programming operating parameters into the microprocessor control;
operating the pump to circulate the cell culture medium through the bioreactor to grow cells or cell-derived products therein; and optionally, carrying out one or both of the following:
harvesting the grown cells or cell-derived products from the bioreactor; and
disposing of said at least one cultureware module.

18. The method of claim 17, wherein said at least one cultureware module includes a gas exchange unit and further comprising the step of providing oxygen and adding or removing carbon dioxide to the cell culture medium to support cell metabolism.

19. The method of claim 17, wherein said at least one cultureware module includes a pH sensor disposed therein and further comprising the step of controlling the pH of the cell culture medium.

20. The method of claim 19, further comprising the step of regulating the cell culture medium feed rate control of the medium.

21. The method of claim 20, wherein the step of regulating the cell culture medium feed rate control includes monitoring carbon dioxide levels in said cell growth chamber to calculate lactate concentration of the cell culture medium.

22. The method of claim 18, further comprising heating said at least one cultureware module to promote cell growth.

23. A method for the production of cells and/or cell-derived products, comprising:
providing at least one least one perfusion bioreactor; introducing cells into said bioreactor, and culturing the cells within said bioreactor, wherein said bioreactor comprises a housing with an inlet port and outlet port, and a cell growth matrix, wherein said cell growth matrix is planar, pleated, or spirally wound around a central core.

24. The method of claim 23, further comprising harvesting the cells, cell-derived product, or both, from said bioreactor.

25. The method of claim 23, wherein the cell-derived product is a virus or viral vector.

26. The method of claim 25, wherein the virus is influenza virus.
